(19) United States
(12) Patent Application Publication
(10) Pub. No.: US 2007/0282317 A1
(43) Pub. Date: Dec. 6, 2007

(54) Implantable Microphone For Treatment Of Neurological Disorders

(76) Inventor: Werner Lindenthaler, Oberperfuss (AT)

Correspondence Address: BROMBERG & SUNSTEIN LLP, 125 SUMMER STREET, BOSTON, MA, 02110-1618, US

(21) Appl. No.: 11/750,572

(22) Filed: May 18, 2007

Related U.S. Application Data
(60) Provisional application No. 60/801,350, filed on May 18, 2006.

(51) Int. Cl. A61B 18/04

(52) U.S. Cl. 606/32

(57) ABSTRACT

A system and method is described for adjunct electrical neuromodulation therapy of urinary incontinence and neurological disorders. In response to a detected event sensed by an implantable microphone, a targeted component is selectively stimulated by electrical stimulation pulses, or a therapeutically effective amount of a selected drug may be delivered to a targeted physiological function location.

This application claims priority from U.S. Provisional Application No. 60/801,350, filed May 18, 2006, and titled “Implantable Microphone For Treatment Of Neurological Disorders,” the contents of which are hereby incorporated by reference.

### FIELD OF THE INVENTION

The invention relates to implants for neurological disorders, and specifically to use of an implantable microphone as an afferent part of an implant for treating neurological disorders.

### BACKGROUND ART

The bladder normally is relaxed while urine collects, and the urethra stays closed. For micturition (urination), the urethra relaxes and intravesical pressure greater than opening pressure is generated. At some threshold point as the bladder fills, the afferent signals of mechanoreceptors in the bladder wall will trigger a coordinated micturition reflex (controlled by a center in the upper pons). The mechanoreceptors are basically tension receptors. The activated mechanoreceptors together with the reflex detrusor muscle in the bladder wall enter a positive feedback loop as micturition begins. Once urine enters the urethra, bladder contraction further responds to reflex excitation from urethral receptors.

The micturition positive feedback loop helps ensure that the bladder empties completely. While fluid remains in the lumen, intravesical pressure stays above the mechanoreceptor threshold, which ensures a continuous driving force for the detrusor. But this mechanism can become unstable so that a passing stimulus may cause impulses in mechanoreceptor afferents that can trigger the micturition reflex. The bladder normally is protected against this happening during the filling by phase spinal and supraspinal safety mechanisms.

One of these spinal safety mechanisms is the reflex control of the striated urethral sphincter. This becomes more active in response to bladder mechanoreceptor activation during the filling phase. This is similar to Edvardsen's reflex of mechanoreceptor activation of sympathetic inhibitory neurons to the bladder. The sympathetic efferents inhibits at the postganglionic neurons in the vesical ganglia, and also at the bladder detrusor muscle. During micturition, the sphincter and sympathetic reflexes normally are turned off at the spinal cord level. There also are inhibitory connections from the cerebral cortex and hypothalamus to the pontine micturition center which act at the supraspinal level.

An over-active bladder and urinary incontinence may be caused by an imbalance between the micturition feedback mechanism and the inhibitory control systems. For example, a hyperexcitable voiding reflex may occur after macroscopic lesions at various nervous system locations, or after minor functional disturbances of the excitatory or inhibitory circuits. Incontinence due to detrusor instability often becomes chronic.

Phasic detrusor instability is one specific type of incontinence in which phasic bladder contractions spontaneously occur during the filling phase. Phasic detrusor instability may also be caused by external stimuli such as rapid filling, coughing, or jumping. This instability is due to imbalance between the micturition feedback and the spinal inhibitory mechanisms.

Another specific type of incontinence is an uninhibited overactive bladder. This may include loss of voluntary micturition control and impaired bladder sensation. Filling sensation is experienced at a normal or lowered volume and is almost immediately followed by involuntary micturition. The urge to go is delayed until voiding has already begun with detrusor contraction and urethra relaxation, at which point, it is too late to voluntarily interrupt the micturition. This is not a malfunction of the bladder mechanoreceptors, as the micturition reflex occurs at normal or even small bladder volumes. Rather, suprapontine sensory projection to the cortex seems to be affected. An uninhibited overactive bladder may occur with neurogenic dysfunction.

One approach to such problems is reinforcement of another inhibitory system. Drug treatment may not fully control the situation. Electro-stimulation can be attempted to improve urethral closure and bladder control. A neuromodulation approach seeks to exploit spinal inhibitory systems that can interrupt a detrusor contraction by electrically stimulating afferent anorectal branches of the pelvic nerve, afferent sensory fibers in the pudendal nerve, and muscle afferents from the limbs.

WO 0027286 describes an “Acoustic Vesicoureteral Reflux Diagnostic System” which is worn on the body as an electronic stethoscope to diagnose vesicoureteral reflux. The stethoscope microphone monitors sound corresponding to a turbulent flow of urine from the bladder backwards from the ureter and towards the kidney. This is similar to the traditional use of a stethoscope as a diagnostic tool placed on the skin to monitor sound within the patient.

### SUMMARY OF THE INVENTION

Embodiments of the present invention are directed to using an implantable sensing microphone to generate a sensing input for controlling a physiological function such as overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation. The sensing microphone generates an electrical signal that is representative of and responsive to activity at an internal sensing location of a user. For example, for controlling urinary function, the microphone might be positioned to sense activity in the lower urinary tract such as the bladder or urethra. In specific embodiments, the microphone may monitor pressure and/or distension changes at the internal sensing location, and/or contraction changes of a targeted muscle at the internal sensing location.

A control unit may be coupled to the microphone, and in response to the microphone signal, may generate a stimulation signal such as a sequence of electrical pulses to electrically stimulate a targeted physiological function location. For example, one or more stimulation electrodes may stimulate inhibitory systems that can interrupt a detrusor contraction by electrically stimulating afferent anorectal branches of the pelvic nerve, afferent sensory fibers in the pudendal nerve, and/or muscle afferents from the limbs.

In addition or alternatively, an implantable drug delivery device may be coupled to the microphone, and in response to the microphone signal may deliver a therapeutically effective amount of a selected drug to a targeted physiological function such as overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation. The sensing microphone generates an electrical signal that is representative of and responsive to activity at an internal sensing location of a user. For example, for controlling urinary function, the microphone might be positioned to sense activity in the lower urinary tract such as the bladder or urethra. In specific embodiments, the microphone may monitor pressure and/or distension changes at the internal sensing location, and/or contraction changes of a targeted muscle at the internal sensing location.

For example, the drug delivery device may be a drug delivery pump arrangement and the embodiment may also include a drug delivery catheter for delivering the selected drug to the target physiological function location. The selected drug may be adapted to stimulate inhibitory systems that can interrupt a detrusor contraction, such as by stimulating afferent anorectal branches of the pelvic nerve, afferent sensory fibers in the pudendal nerve, and/or muscle afferents from the limbs.

In specific embodiments, the targeted physiological function location includes an afferent function and/or an efferent function. And the embodiment may further be responsive to user control in generating the stimulation signal and/or drug delivery signal. The sensing microphone may specifically monitor pressure changes at the internal sensing location and/or contraction changes of a target at the internal sensing location.

### BRIEF DESCRIPTION OF THE DRAWINGS

FIG. 1 shows one embodiment of the present invention in which a sensing microphone is integrated into the body of an implantable control unit.

FIG. 2 shows another embodiment in which a sensing microphone is physically separate from the implantable control unit.

FIG. 3 shows an example of an embodiment such as the one in FIG. 2 as implanted to monitor and control the functioning of the lower urinary tract.

### DETAILED DESCRIPTION OF SPECIFIC EMBODIMENTS

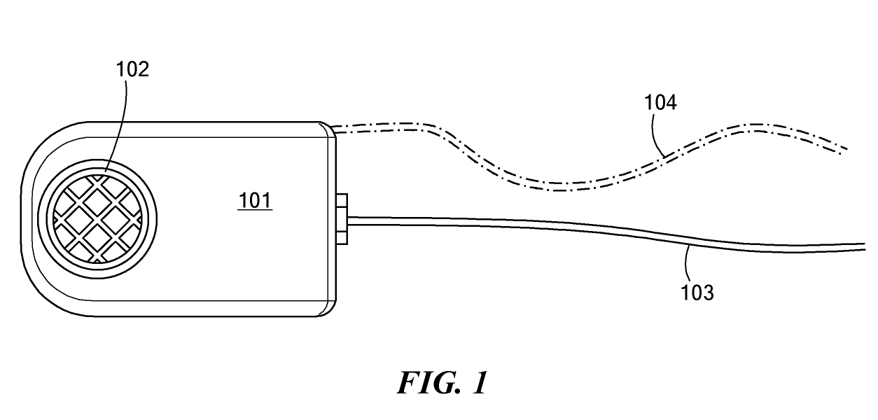

FIG. 1 shows an example of an implantable physiological function control system according to one specific embodiment of the present invention. Implantable control unit 101 contains a built-in sensing microphone 102. The control unit 101 is implanted so that the microphone is able to sense pressure, distension and/or contraction activity at a target internal sensing location such as the wall of the patient's bladder in the lower urinary tract. Other physiological functions that may usefully be monitored and/or controlled include without limitation overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation.

The microphone senses such activity and generates a representative electrical signal for the control unit 101. Rather than a microphone as such, some embodiments may use other similar types of sensor such as, without limitation, a piezoelectric pressure sensor or other pressure sensor. In response, the control unit may generate a stimulation signal for electrode array 103 to electrically stimulate a targeted location such as the patient's urethra. For example, the stimulation signal may be a sequence of electrical pulses for the electrodes to stimulate the target location. In addition, or alternatively, the control unit 101 may use an implanted drug delivery catheter 104 to deliver a therapeutically effective amount of a selected drug to a target location such as the patient's urethra. In specific embodiments, the operation of the control unit 101 may be responsive volitional control of the patient.

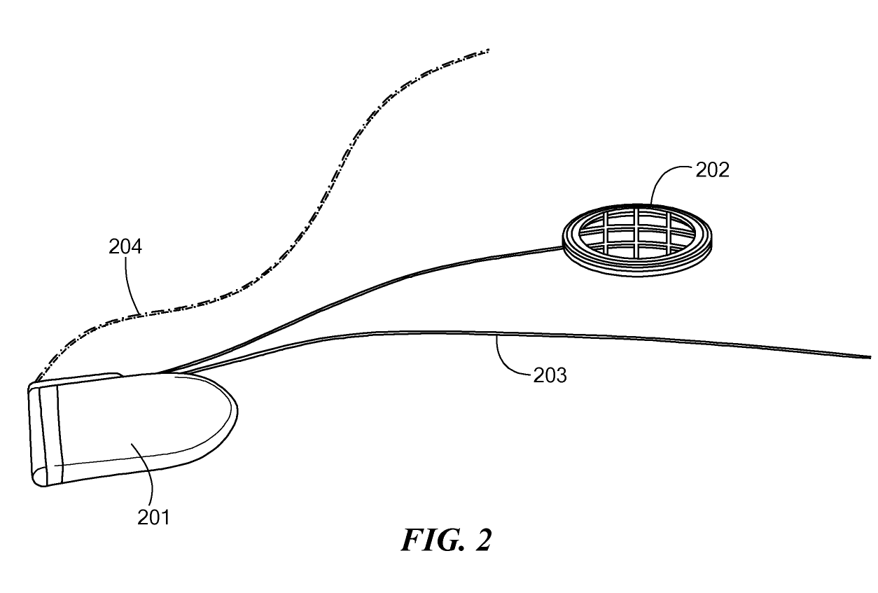

FIG. 2 shows an alternative embodiment in which an implantable sensing microphone 202 is physically separate from the control unit 202. This allows for sensing microphone 202 to placed at an sensing location which is optimal for detecting the target activity, while the control unit 201 can be implanted at a different location which may be more convenient in terms of its bulk and positioning, and may for example, allow for more convenient post-implantation servicing of the control unit 201 without disturbing the sensing microphone 202 and its location.

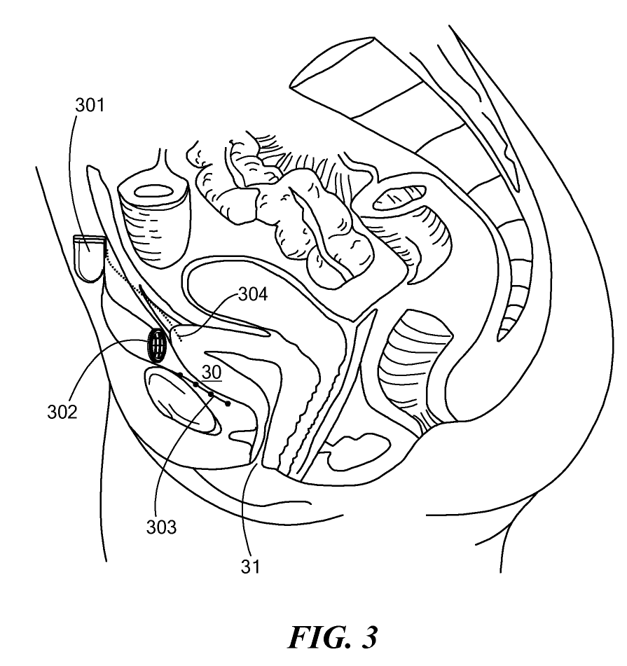

FIG. 3 shows an example of an implanted system such as the one shown in FIG. 2 for monitoring and controlling the patient's bladder 30 and urethra 31. In FIG. 3, an implantable sensing microphone 302 is located near the wall of the patient's bladder 30 and senses activity therein. A representative electrical signal from the sensing microphone 302 is coupled to a control unit 301 which acts responsively. For example, the control unit 301 may generate a stimulation signal for implanted electrode array 303 to stimulate the bladder 30 and/or the urethra 31. In addition or alternatively, the control unit 301 may cause implanted drug delivery catheter 304 to deliver a therapeutically effective amount of one or more selected drugs to a target location such as the interior volume of the bladder 30.

In specific embodiments, the targeted physiological function location may be an afferent function such as a nerve sensing location, and/or an efferent function such as a motor nerve location. Specific embodiments may seek to exploit spinal inhibitory systems that can interrupt a detrusor contraction by electrically stimulating afferent anorectal branches of the pelvic nerve, afferent sensory fibers in the pudendal nerve, and/or muscle afferents from the limbs.

Although various exemplary embodiments of the invention have been disclosed, it should be apparent to those skilled in the art that various changes and modifications can be made which will achieve some of the advantages of the invention without departing from the true scope of the invention.

What is claimed is:

1. A system for controlling a physiological function comprising:
an implantable sensing microphone for generating an electrical signal representative of activity in an internal sensing location of a user; and
a control unit coupled to the microphone and responsive to the microphone signal for generating a stimulation signal to electrically stimulate a targeted physiological function location.

2. A system according to claim 1, wherein the stimulation signal includes a sequence of electrical pulses.

3. A system according to claim 1, further comprising:
at least one implantable stimulation electrode for applying the stimulation signal to the targeted physiological function location.

4. A system according to claim 3, wherein the electrode is adapted to stimulate an inhibitory system capable of interrupting a detrusor contraction.

5. A system according to claim 4, wherein the inhibitory system includes one of anorectal branches of the pelvic nerve, afferent sensory fibers in the pudendal nerve, and muscle afferents from the limbs.

6. A system according to claim 1, wherein the targeted physiological function location includes an afferent function.

7. A system according to claim 1, wherein the targeted physiological function location includes an efferent function.

8. A system according to claim 1, wherein the targeted physiological function location includes an afferent function and an efferent function.

9. A system according to claim 1, wherein the control unit is further responsive to user control in generating the stimulation signal.

10. A system according to claim 1, wherein the physiological function includes at least one of overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation.

11. A system according to claim 1, wherein the microphone is located in the lower urinary tract of the user.

12. A system according to claim 1, wherein the microphone monitors pressure changes at the internal sensing location.

13. A system according to claim 1, wherein the microphone monitors contraction changes of a target at the internal sensing location.

14. A system according to claim 1, wherein the microphone monitors distension changes of a target at the internal sensing location.

15. A system for controlling a physiological function comprising:
an implantable sensing microphone for generating an electrical signal representative of activity in an internal sensing location of a user; and
an implantable drug delivery device coupled to the microphone and responsive to the microphone signal for delivering a therapeutically effective amount of a selected drug to a targeted physiological function location.

16. A system according to claim 15, further comprising:
a drug delivery catheter for delivering the selected drug to the target physiological function location.

17. A system according to claim 15, wherein the drug delivery device is an implanted pump.

18. A system according to claim 15, wherein the delivered drug is adapted to stimulate an inhibitory system capable of interrupting a detrusor contraction.

19. A system according to claim 18, wherein the inhibitory system includes one of anorectal branches of the pelvic nerve, afferent sensory fibers in the pudendal nerve, and muscle afferents from the limbs.

20. A system according to claim 15, wherein the targeted physiological function location includes an afferent function.

21. A system according to claim 15, wherein the targeted physiological function location includes an efferent function.

22. A system according to claim 15, wherein the targeted physiological function location includes an afferent function and an efferent function.

23. A system according to claim 15, wherein the control unit is further responsive to user control in delivering the selected drug.

24. A system according to claim 15, wherein the physiological function includes at least one of overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation.

25. A system according to claim 15, wherein the microphone is located in the lower urinary tract of the user.

26. A system according to claim 15, wherein the microphone monitors pressure changes at the internal sensing location.

27. A system according to claim 15, wherein the microphone monitors contraction changes of a target at the internal sensing location.

28. A system according to claim 15, wherein the microphone monitors distension changes of a target at the internal sensing location.

29. A system for monitoring a physiological function comprising:
an implantable sensing microphone for generating an electrical signal representative of activity in an internal sensing location of a user; and
a control unit coupled to the microphone.

30. A system according to claim 29, wherein the microphone is located in the lower urinary tract of the user.

31. A system according to claim 29, wherein the microphone monitors pressure changes at the internal sensing location.

32. A system according to claim 29, wherein the microphone monitors contraction changes of a target at the internal sensing location.

33. A system according to claim 29, wherein the microphone monitors distension changes of a target at the internal sensing location.

34. A system according to claim 29 wherein the activity includes an afferent function.

35. A system according to claim 29, wherein the activity includes an efferent function.

36. A system according to claim 29, wherein the activity includes an afferent function and an efferent function.

37. A system according to claim 29, wherein the control unit is further responsive to user control.

38. A system according to claim 29, wherein the activity includes at least one of overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation.

39. A system according to claim 29, wherein the microphone is located in the lower urinary tract of the user.
